(19) United States
(12) Patent Application Publication
(10) Pub. No.: US 2025/0281939 A1
(43) Pub. Date: Sep. 11, 2025

(54) LEAK-PROOF ATOMIZING ASSEMBLY AND ATOMIZING EQUIPMENT

(71) Applicant: Guangdong Baixiang Environmental Technology Co., Ltd., Guangzhou (CN)

(72) Inventor: Bing Yao, Guangzhou (CN)

(21) Appl. No.: 19/011,464

(22) Filed: Jan. 6, 2025

(30) Foreign Application Priority Data
Mar. 7, 2024 (CN) 2024102582725

(51) Int. Cl. B05B 7/26; B05B 7/24

(52) U.S. Cl. CPC B05B 7/262; B05B 7/2491

(57) ABSTRACT

A leak-proof atomizing component, which comprises an atomizing head and a atomizing core; The atomizing head is provided with a liquid return hole that is connected with the peripheral bottle body; When the return liquid hole is only one, the diameter of the return liquid hole is the first preset value, so that the liquid in the peripheral bottle cannot continuously flow out from the return liquid hole when the peripheral bottle is poured; When the return liquid hole is multiple, the diameter of the return liquid hole is the second preset value, so that when the peripheral bottle body is poured, the liquid in the peripheral bottle body cannot continuously flow out of the return liquid hole due to the existence of liquid tension. By optimizing the return hole, the component avoids continuous liquid out flow through the return hole when the bottle is poured.

### FIELD OF TECHNOLOGY

The invention belongs to the spray field, in particular to a leak-proof atomizing component and atomizing equipment.

### BACKGROUND TECHNOLOGY

Oil leakage is a problem that has plagued technicians in this field for a long time. The continuous leakage of essential oils will not only waste essential oils, but also enter the electronic control components to corrode the circuit board or damage the air pump, resulting in equipment damage and if seriously lead to a fire.

To solve this problem, related art proposes the following solutions:

Related art 1: CN116585517A discloses an oil-proof diffuser, which blocks the oil leakage in the oil bottle by means of a support. The utility model discloses that it contains at least one return pipe, and the diameter of the return pipe is greater than the straight diameter of the suction pipe. There is a lot of liquid in the return pipe. When there is too much liquid in the leak proof parts, the slightly inclined liquid leaks out to the outside of the equipment, which cannot be completely eliminated but can only play a slowing role, especially in the fixed Angle, which cannot be prevented at all, and the unique Angle will play a slowing role.

The problem is that the support can only block a part of the essential oil, if there are more essential oils in the bottle so that the essential oil can not live in the return oil pipe when the bottle is poured, and the diameter of the return oil pipe is large, the essential oil will continue to leak into the atomizing chamber with the return oil pipe.

Related art 2: CN114832965A discloses an anti-tipping oil diffuser which, by means of a drive assembly, seals the spray position when the bottle is tipped to prevent oil leakage.

It is effective to block the spray port through the drive component, but it is necessary to add a structure that requires external power or seal through the elastic component, and the overall structural complexity will be significantly increased.

Related art 3: CN112336903A discloses a leak proof anhydrous essential oil diffuser, which is similar to related art 2 in that the spray nozzle is closed when pouring with a drive assembly.

In the existing technology, in order to prevent oil leakage, most of them focus on how to close the fog outlet; However, the result of this effort often leads to the equipment structure is too complex, so that the injection molding process is difficult to achieve product production.

Therefore, the technical problem solved in this case is: how to achieve leakage prevention of spray equipment without basically changing the basic structure of the existing equipment.

### Contents Of The Invention

One of the purposes of the invention is to provide a leak-proof atomizing component which, by optimizing the liquid return hole, can avoid the continuous outflow of liquid through the liquid return hole when the bottle body is poured.

At the same time, the invention also discloses a atomizing device.

To realize the above purpose, the invention provides a leak-proof atomizing component, including a atomizing head for connecting with a peripheral bottle body; The atomizing head is provided with an atomizing core for atomizing the liquid in the bottle and producing spray: The atomizing head is provided with a liquid return hole that is communicated with the peripheral bottle body;

Except the atomizing core, the liquid return hole is the only channel for the peripheral bottle body to communicate with the external atmosphere;

The liquid return hole is one or more.

When the return liquid hole is one, the diameter of the return liquid hole is the first preset value, so that the liquid in the peripheral bottle can not continuously flow out of the return liquid hole when the peripheral bottle is tipped over;

When the liquid return hole is multiple, the diameter of the liquid return hole is the second preset value, so that when the peripheral bottle body is tipped over, the liquid in the peripheral bottle body cannot continuously flow out of the liquid return hole due to the existence of liquid tension.

In this field, the atomizing head refers to a conventional component connected with the bottle body, which is equipped with an atomizing core for the liquid in the bottle body under the action of compressed gas suction, atomization;

The liquid return hole (also known as the oil return hole in some existing technology) is also a conventional design, the purpose of setting the liquid return hole is generally used to make the larger liquid drop back to the atomizer head can smoothly enter the bottle, avoid liquid oxidation, and the liquid return hole also plays the role of balancing the pressure balance inside and outside the bottle.

For the existing research in this field, it is generally believed that the return hole should be set up larger, so that the liquid can smoothly return to the bottle.

In the ongoing research process, we found that if the diameter of the return hole is controlled to a certain size and the number of return holes is set at a specific size, the oil leakage can be slowed, while still ensuring that the liquid can be returned to the bottle under the negative pressure.

For those skilled in this field, the basic principle of oil return is to return oil based on gravity factors. As one of the core research results of this case, we found that even if the diameter of the liquid return hole is small, it is still possible to return oil, because if the oil is returned, it means that the spray is in progress, and the liquid in the bottle will continue to decrease during the spray process. Then the bottle will inevitably form negative pressure, and then the liquid outside the return hole will be sucked into the bottle; Based on the research, the invention is optimized as follows:

For example, when the liquid return hole is one, when the diameter of the liquid return hole is reduced to a certain extent, the liquid can not form a liquid channel and a gas channel coexist in the liquid return hole, and the liquid has a certain surface tension in the liquid return hole, when the bottle is dumped and the equipment is not working, the liquid will drown the liquid return hole, and the liquid may flow out for a short time. When there is a certain pressure difference between the inside and outside of the bottle, the liquid cannot flow out sustainably; Of course, when the equipment continues to work and the bottle is poured, it is more unlikely to leak, because the atomizing head continues to spray liquid, which will cause the liquid return hole to constantly inhale the outside world into the bottle, and the liquid in the bottle naturally cannot continue to flow out.

For example, when there are more than one return hole, when the diameter of the return hole is smaller, the liquid in the return hole will produce a very large surface tension, so that the liquid cannot continue to flow out.

It can be seen that through the above optimization, the basic function of the liquid return hole can be maintained-reflux liquid, maintain the balance of the pressure inside and outside the bottle, and at the same time, the continuous leakage of the liquid in the bottle can be avoided when the bottle is topple over.

In the leak-proof atomizing component, the first preset value is less than or equal to 2 mm, and, further optimally, the first preset value is less than or equal to 1.5 mm, further optimally, the first preset value is 0.2˜1.2 mm, and more optimally, the first preset value is 0.5˜1 mm.

In some implementation embodiment of the invention, The first preset value is 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm or 2 mm;

It should be noted that because the viscosity of different liquids is different, the first preset value in the invention is changeable to a certain extent. If the viscosity of the liquid is large, the first preset value can be appropriately increased, and if the viscosity of the liquid is small, the first preset value can be appropriately reduced.

In the leak-proof atomizing components assembly, the second preset value is less than 0.2 mm.

In some embodiment of the invention, if the number of liquid return holes is multiple, then the number can be selected to be more than 2, preferably 2 to 20, preferably 2 to 10; For example, it can be 2, 3, 4, 5, 7, 9, 10, 15, 20 or more; Its diameter needs to be designed smaller, generally speaking, it can be 0.05 mm, 0.1 mm, 0.12 mm, 0.15 mm, 0.16 mm, 0.17 mm, 0.18 mm, 0.19 mm; Smaller diameters are not only more expensive to process, but also easier to clog, so they are not listed.

In the leak-proof atomizing assembly, the atomizing head is provided with an atomizing chamber; The atomizing head is provided with a fog outlet, and the atomizing chamber is conductive to the atmosphere through the fog outlet

The function of the atomizing chamber is to provide a space to accommodate the spray, and part of the role of the space is to provide a buffer space for the large straight diameter of the liquid drop, so that it can fall back into the atomizing head and enter the bottle through the return hole.

In the leak-proof atomizing assembly, the atomizing chamber is provided with an atomizing cover; The atomizing cover divides the atomizing chamber into two connected chambers, and the atomizing mouth and the atomizing core are located on both sides of the atomizing cover.

The existence of atomizing cover can further improve the probability of falling down of large diameter droplets, so that the liquid diameter of the mist emitted through the fog outlet is smaller and the particle size distribution is more uniform.

In some designs in this field, some blocking structures such as mazes can also be set on the atomizing cover to prolong the path of the spray from the atomizing chamber to the fog outlet, and further improve the probability of falling down the droplets with larger diameters;

Some holes can be arranged on the atomizing cover to realize the diffusion of fog from the atomizing chamber to the fog outlet; The number of holes is generally one, but can also be multiple;

In the leak-proof atomizing assembly, the atomizing head is provided with an atomizing seat, and the atomizing core is fixed on the atomizing seat; The atomizing head and the atomizing seat are provided with a gas channel and a liquid channel that lead to the atomizing core; The liquid channel is connected to the peripheral bottle body through a pipeline; The gas channel is connected with a peripheral gas source.

In the leak-proof atomizing components assembly, the liquid return hole is connected with a conduit extending to the middle or lower part of the peripheral bottle body. In some application cases of the invention, the catheter can also be extended to the upper part of the bottle body; In any respect, the existence of the conduit has no absolute effect on the realization of the purpose of the invention; Only from the optimal point of view, the catheter extends to the middle and lower part of the bottle, and after the bottle is dumped, the catheter will be out of the liquid level with a high probability to further eliminate the phenomenon of leakage.

In the leak-proof atomizing assembly, the atomizing head is provided with a concave part on one side of the peripheral bottle body; The liquid return hole is arranged in the concave part.

Based on the dead weight of the liquid, the larger diameter droplets fall back into the atomizer head, and they will converge to the concave. During the operation of the atomizer head, the negative pressure generated in the bottle body will draw these liquids into the bottle body; Avoid residual liquid in atomizing head.

In the leak-proof atomizing assembly, the atomizing core is arranged in the concave part.

In some embodiment of the invention, the location of the atomizing core is very diversified, such as the middle and upper part of the atomizing chamber, the middle and lower part of the atomizing chamber, and so on; The spray direction of the atomizing core can be upward, downward, or sideways spray;

The advantage of atomizing core set in the concave is that it can provide the probability of collision between the spray and the wall of the concave, further improve the probability of falling down of the larger diameter droplets, and more effectively collect the liquid with larger particle size and return to the bottle.

In the leak-proof atomizing assembly, the liquid return hole is arranged at the lowest part of the concave part.

In the leak-proof atomizing assembly, the top of the concave part is arranged at the lowest of the corresponding surface of the atomizing head.

In the leak-proof atomizing assembly, the atomizing head is provided with a stop ring; The stop ring is arranged on a path where the liquid flows from the return liquid hole to the exit fog gate.

In the leak-proof atomizing assembly, the baffle ring is arranged on the inside of the atomizing head and near the fog outlet.

In the leak-proof atomizing component, the stop ring is protruded inward to form a round table; The fog outlet is located in the center of the circular platform; The half section of the retaining ring and the half section of the top wall of the atomizing head constitute a “7” font structure.

At the same time, the invention also discloses a atomizing device, which comprises a leak-proof atomizing component, a bottle body and an air source mentioned in any of the above items; The atomizing head is fixedly connected with the bottle body; The bottle body is used to provide the liquid required for spraying to the atomizing core; The gas source is used to provide compressed gas required for spraying to the atomizing core.

The atomizing equipment also comprises a shell in which the bottle body is placed.

In the atomizing equipment, the air source is placed in the housing and located below the bottle body.

In the atomizing equipment, the bottom of the shell is also provided with a base; The base is provided with an electronic control assembly for controlling the gas source.

In the atomizing equipment, the liquid in the bottle is an essential oil or a disinfectant or mosquito repellent used to produce a fragrance.

Compared with the related art, the invention has the following beneficial effects:

By optimizing the liquid return hole, the invention can avoid the continuous liquid flow out through the liquid return hole when the bottle body is poured.

When the liquid return hole is one, when the diameter of the liquid return hole is reduced to a certain extent, the coexistence of liquid body channel and gas channel cannot be formed in the liquid return hole. At the same time, the liquid has a certain surface tension in the liquid return hole. When the bottle is dumped and the equipment is not working, the liquid will drown the liquid return hole, and the liquid may flow out for a short time. The liquid cannot flow out sustainably: Of course, when the equipment is continuously working and the bottle is poured, it is more unlikely to leak, because the continuous spraying of liquid by the atomizer head will cause the liquid return hole to constantly inhale the outside world into the bottle, and the liquid in the bottle naturally cannot continue to flow out.

When there are more than one return hole, when the diameter of the return hole is smaller, the liquid in the return hole will produce a very large surface tension, so that the liquid cannot be sustained.

Through the above optimization, the basic function of the liquid return hole can be maintained—to return the liquid, maintain the balance of the pressure inside and outside the bottle, and at the same time, it can avoid the continuous leakage of the liquid in the bottle when the bottle is tipped.

### INSTRUCTION WITH FIGURES

FIG. 1: implementation 1 and 3 of the invention relate to stereoscopic drawings of atomizing equipment;

FIG. 2: Implementation 1 and 3 of the invention relate to a section view of the atomizing equipment;

FIG. 3: A top view of the body of implementation 1 of the invention

FIG. 4: Stereo gram of the body of implementation 1 of the invention;

FIG. 5: A section view of the body of implementation 1 of the invention;

FIG. 6: A stereo gram of the body of implementation 1 of the invention;

FIG. 7: The main view of another atomizing device referred to in implementation 1 and implementation 3 of the invention;

FIG. 8: Implementation 1 and 3 of the invention relate to a stereo gram of another atomizing equipment;

FIG. 9: Implementation 1 and 3 of the invention relate to a section view of another atomizing device;

FIG. 10: A stereo gram of the body in FIG. 9 of the invention;

FIG. 11: Implementation 1 and 3 of the invention relate to a stereo gram of another atomizing device

FIG. 12: Implementation 1 and 3 of the invention relate to a section view of another atomizing device;

FIG. 13: A stereo gram of the body in FIG. 12 of the invention

FIG. 14: Implementation 2 of the invention has a top view of the body.

### SPECIFIC IMPLEMENTATION MODE

The technical scheme of the invention is further explained by means of specific implementation. It should be clear to those skilled in the art that the implementation are merely intended to assist in the understanding of the invention and should not be regarded as specific limitations of the invention.

### Implementation 1

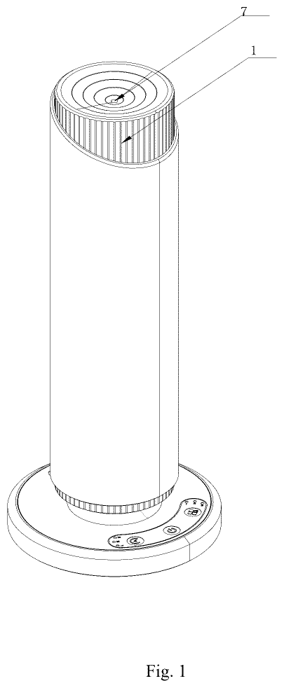

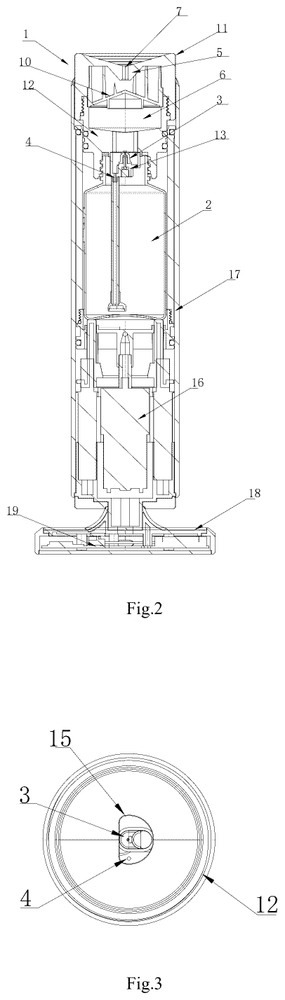

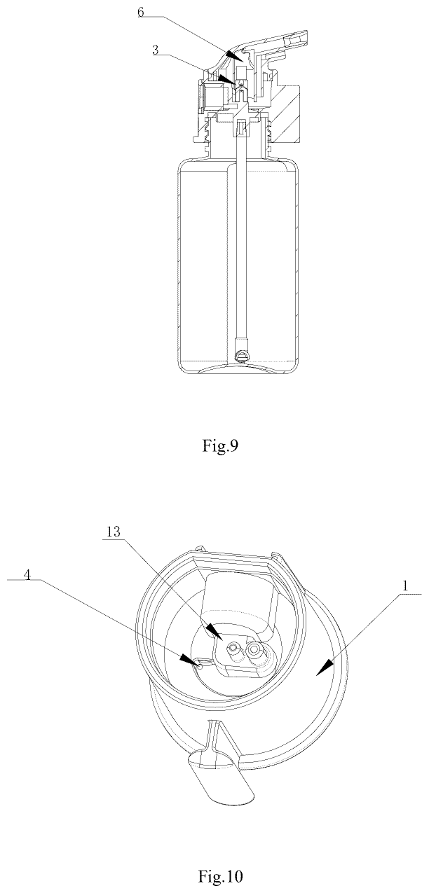

Refer to FIGS. 1-13:

A leak-proof atomizing assembly comprises an atomizing head 1 for connecting with a peripheral bottle body 2; The atomizing head 1 is provided with an atomizing core 3 for atomizing the liquid in the bottle body 2 and producing spray; The atomizing head 1 is provided with a liquid return hole 4 that is communicated with the peripheral bottle body 2, and the liquid return hole 4 is the only channel for the peripheral bottle body 2 to communicate with the external atmosphere;

The liquid return hole 4 is one, and the diameter of the liquid return hole 4 is the first preset value, so that the liquid in the peripheral bottle body 2 cannot continuously flow out of the liquid return hole 4 when the peripheral bottle body 2 is poured.

When the diameter of the liquid return hole 4 is reduced to a certain extent, the coexistence of liquid channel and gas channel cannot be formed in the liquid return hole 4. At the same time, the liquid has a certain surface tension in the liquid return hole 4. When the bottle body 2 is dumped and the equipment is not working, the liquid will drown the liquid return hole 4, and the liquid may flow out for a short time. There will be a pressure difference inside and outside the bottle, and when there is a certain pressure difference inside and outside the bottle, the liquid cannot continue to flow out; Of course, when the equipment is continuously working and the bottle 2 is poured, it is even less likely to leak, because the continuous spraying of liquid by the atomizer head 1 will cause the liquid return hole 4 to constantly inhale the outside world into the bottle 2, and the liquid in the bottle 2 will naturally not be sustainable.

Even if the liquid return hole 4 is small to a certain extent, it still will not block the liquid return, the reason is that the atomizing head 1 will continuously suck out the liquid in the bottle body 2 during the spray process; If the return hole 4 is covered by liquid, the gas pressure in the bottle body 2 will be reduced, and the atmosphere will press the liquid near the return hole 4 into the bottle body 2, achieving a smooth return of the liquid.

Refer to FIGS. 1-6:

In the present implementation, the atomizing head 1 is divided into two parts, the upper part is the upper cover 11, and the lower part is the body 12; The upper cover 11 is sealed connected to the body 12; In the field of power, the commonly used sealing connection methods can be snap fit, inserted, threaded connection, etc. In the present embodiment, the upper cover 11 and the body 12 are connected by threads; The spray outlet is arranged on the upper cover 11, and the liquid return hole 4 is arranged on the body 12.

In this implementation, the first default value for achieving the above purpose is generally less than 2 mm; In several tests of the invention, we verify that 1.9 mm, 1.5 mm, 1 mm, 0.9 mm, 0.8 mm and liquid return holes with a diameter lower than 0.8 mm can avoid the continuous leakage of liquid when the bottle body 2 is poured;

As other realizable means of the present implementation, the basic purpose of the invention may be realized only by requiring body 12, that is, by ensuring that it can be sprayed into the atmosphere. In practical applications, having an upper cover 11 or a covering such as a fogging cover 10 above the body 12 is the most common form.

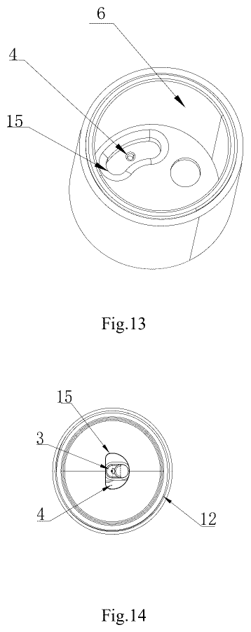

There are many ways to implement ontology 12, as shown in FIG. 5, FIG. 10 and FIG. 13.

In this implementation, the upper cover 11 and the body 12 are obtained by injection molding; In the process of installation, first install the atomizing core 3 on the body 12, and then tighten the upper cover 11; Body 12 and bottle 2 are securely connected.

As a further refinement of the implementation, the atomizing head 1 is provided with an atomizing cavity 6; The atomizing head 1 is provided with a fog outlet 7, and the atomizing chamber 6 is communicated through the fog outlet 7 and the atmosphere.

The function of the atomizing chamber 6 is to provide a space to accommodate the spray, and part of the function of the space is to provide a buffer space for the large-diameter droplets, so that they can fall back into the atomizing head 1 and enter the bottle body 2 through the liquid return hole 4.

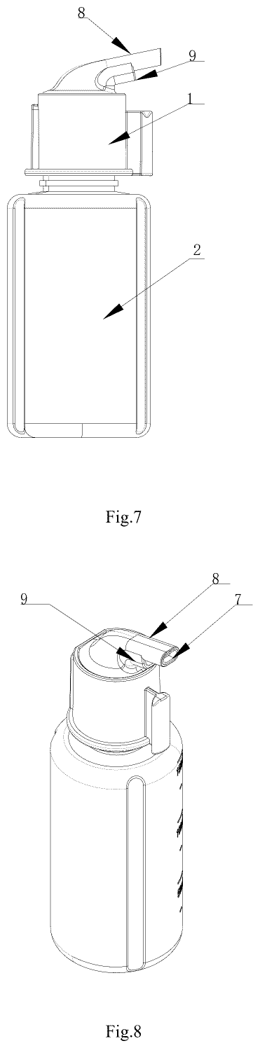

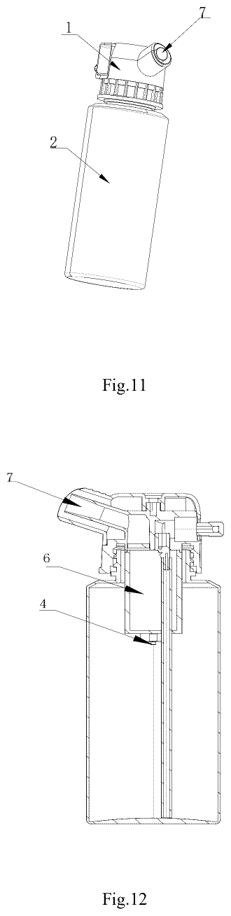

The existence of atomizing chamber 6 means that there is no obvious limitation on the direction of atomizing head 1. In the atomizing chamber 6, atomizing head 1 can spray upward (one realization form of FIG. 1-6 and one realization form of FIG. 7-10), downward spray and side spray (An implementation of FIG. 11-FIG. 13);

The atomizing chamber 6 can be outside the bottle body 2, and is generally chosen to be above the bottle body 2; The atomizing chamber 6 can also be extended from the mouth of the bottle to the inside of the bottle body 2, as shown in FIG. 12

As the optimization of the invention, the design forms of the fog outlet 7 can be various, as shown in FIG. 1-FIG. 6, which is arranged on the top of the atomizing head 1; It can also be shown in FIGS. 7 and 11, which extend outward at an oblique Angle through an inclined fog pipe 8; In order to further improve the delivery distance of the spray, a inlet air pipe 9 can also be added to the outside of the spray pipe 8, and the high-speed compressed gas generated by the inlet air pipe 9 can further increase the delivery distance of the spray. High-speed compressed gas can optionally be generated from a peripheral gas source.

In some preferred cases of the present embodiment, the atomizing head 1 is provided with a stop ring 5; The baffle ring 5 is arranged on the path of the liquid flowing from the return liquid hole 4 to the exit fog gate 7. In general, the baffle ring 5 has a height of 0.5˜2 cm, and its main purpose is that when the bottle body 2 is inverted, it can block a small amount of liquid in the atomizing chamber 6 from the fog outlet 7.

More optimally, the stop ring 5 is arranged on the inside of the atomizing head 1 and near the fog outlet 7.

More optimally, the atomizing cavity 6 is provided with an atomizing cover 10; The atomizing cover 10 divides the atomizing chamber 6 into two connected chambers, and the atomizing outlet 7 and the atomizing core 3 are located on both sides of the atomizing cover 10.

The presence of atomizing cover 10 can further improve the probability of falling back of droplets with larger diameters, so that the liquid diameter of the mist emitted through the fog outlet 7 is smaller and the particle size is more uniform;

In some designs in this field, the structure of some blocking functions such as maze can also be set on the atomizing cover 10, as shown in FIG. 2, so as to prolong the path of the spray from the atomizing chamber 6 to the fog outlet 7, and further improve the falling rate of droplets with larger diameter.

Some holes can be arranged on the atomizing cover 10 to realize the diffusion of fog from the atomizing chamber 6 to the fog outlet 7; The number of boles is generally one, can also be multiple;

In the present embodiment, the atomizing head 1 is provided with an atomizing seat 13, and the atomizing core 3 is fixed on the atomizing seat 13; The atomizing head 1 and the atomizing seat 13 are provided with a gas channel and a liquid channel that lead to the atomizing core 3; The liquid channel is connected to a peripheral bottle body 2 through a pipeline; The gas channel is connected with a peripheral gas source.

Atomizing head 1 is a conventional design in the field. This embodiment does not restrict a specific atomizing head 1. Any atomizing head 1 capable of producing spray is applicable to the invention.

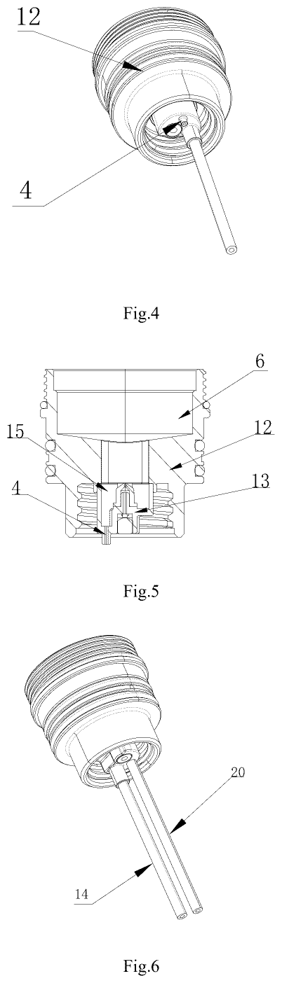

As a further preferred option for this embodiment, reference is made to FIG. 6 where the liquid return hole 4 is connected with a catheter 14 extending to the middle or lower part of the peripheral bottle body 2.

The conduit 14 May be independently extended into the bottle body 2, or may be connected by an additional pipe and a pipe connected to the liquid passage; The advantage of its design is that during the spray process, the suction generated by the liquid channel will make the inlet of the guide tube 14 have greater negative pressure, which is conducive to the reflux of the liquid.

As a further optimization of the embodiment, the atomizing head 1 is provided with a concave part 15 on one side near the peripheral bottle body 2; The liquid return hole 4 is arranged in the concave part 15.

Based on the dead weight of the liquid, the larger diameter droplets will converge to the concave 15 after falling back into the atomizer head 1. When the atomizer head 1 is working, the negative pressure generated in the bottle body 2 will draw these liquids into the bottle body 2. Avoid residual liquid in atomizing head 1.

Preferably, the atomizing core 3 is arranged in the concave part 15. In some implementation of the invention, the location of atomizing core 3 is very diversified, such as the middle and upper part of atomizing chamber 6, the middle and lower part of atomizing chamber 6, and so on;

The advantage of atomizing core 3 set in concave 15 is that it can provide the probability of collision between the spray and the wall of concave 15, and further improve the probability of falling back of larger diameter droplets, which can more effectively collect liquid with larger diameter and return to the bottle.

Further optimally, the liquid return hole 4 is arranged at the lowest part of the concave part 15, which facilitates the accumulation of liquid.

Further optimally, the top of the concave part 15 is arranged at the lowest of the corresponding face of the atomizing head 1.

### Functional Test

According to Embodiment 1, the invention tests whether there is oil leakage after dumping under different apertures of the liquid return hole;

The viscosity of the essential oil was 9.5 MPA·S. The indoor temperature is 25° C., and the air humidity is 45%.

The relevant test parameters and data are shown in Table 1. The height of the liquid in the bottle is 70% of the height of the bottle; The test state of oil leakage is horizontal dumping in shutdown state, and the leakage of the return liquid hole is observed; At the same time, the oil return smoothness of the liquid return hole was tested under the spray state.

TABLE 1
Test parameters and results

| | | | | Oil return |
|---|---|---|---|---|
| | | | | smoothness |
| ffirst | | | | in spray |
| preset | Oil leakage situation | | | condition |
| value | 1 min | 5 min | 10 min | smooth |
| 0.2 | no leakage | no leakage | no leakage | smooth |
| 0.5 | no leakage | no leakage | no leakage | smooth |
| 0.8 | no leakage | no leakage | no leakage | smooth |
| 1 | no leakage | no leakage | no leakage | smooth |
| 1.2 | no leakage | slight leakage | compare to 5 min, | smooth |
| | | leakage volume | leakage volume | |
| | | no increase | no increase | |
| 1.5 | slight | slight leakage | slight leakage | smooth |
| | leakage | leakage volume | leakage volume | |
| | | little increase | little increase | |
| 2 | slight | compare to 1 min, | compare to 5 min, | smooth |
| | leakage | leakage volume | leakage volume | |
| | | obviously | obviously | |
| | | increase | increase | |
| 3 | Continuous | Continuous | Continuous | smooth |
| | leakage | leakage | leakage | |
| 4 | Continuous | Continuous | Continuous | smooth |
| | leakage | leakage | leakage | |

The above test object is the viscosity of the most commonly used essential oil in the production process, in some application scenarios, we will use an essential oil with a larger viscosity than the essential oil, in this scenario, 1. The leakage state of 5 mm aperture and 2 mm aperture will be significantly improved.

### Implementation 2

It is roughly the same as implementation 1, but the difference is that, as shown in FIG. 14, the above-mentioned liquid return holes 4 are multiple. In the actual experiment, we experimented with 3, 4 and 10 holes, and the experiment diameter was 0.2 mm and 0.1 mm. All of them can prevent the outflow of liquid in the bottle 2; Even if part of the liquid return hole 4 is permeable to the atmosphere, the other part of the liquid return hole 4 that is not below the liquid level will still be unable to leak liquid continuously due to the too small aperture and too large capillary surface tension.

For those skilled in the field, a smaller diameter means a larger processing cost, and there is no doubt that the liquid does not leak under the conditions of the above diameter, and the liquid is less likely to leak in the case of a smaller diameter.

In some preferred cases of the invention, the liquid return bole 4 May be centrally arranged in one location or distributed at various locations on the wall of the atomizing head 1. It should be noted that we recommend that the above liquid return hole 4 be set at the same level and high degree, and it is better to concentrate on a lower position, so as to ensure that during the suction process, the liquid in the atomizing cavity 6 can simultaneously flood all the liquid return hole 4, so as to ensure a certain negative pressure in the bottle body 2, so as to achieve the effect of suction and rapid return flow.

### Implementation 3

Refer to FIG. 1-13, which shows at least three types of atomizing equipment, including the leak-proof atomizing assembly, bottle body 2, and air source described in Embodiment 1 or Embodiment 2; The atomizing head 1 is fixedly connected with the bottle body 2; The bottle body 2 is used to provide the liquid required for spraying to the atomizing core 3; The air source is used to provide compressed gas required for spraying to atomizing core 3. The gas source is a gas pump 16 or a compressed gas cylinder; In practical applications, air pump 16 is preferred; The liquid channel in atomizing head 1 is extended to bottle body 2 through pipe 20; The pipe extends below the liquid level of the liquid in bottle 2.

Preferably, referring to FIGS. 1 and 2, the equipment also includes a housing 17, the bottom of which is provided with a base 18; The bottle body 2 is arranged in the shell 17, the air source is arranged in the shell 17 and is located under the bottle body 2, and the base 18 is provided with an electronic control assembly 19 for controlling the air source.

In this implementation, it is preferred to separate the base 18 and the shell 17, and centrally arrange the electronic control component 19, such as the circuit board, in the base 18. The base 18 is kept sealed by sealing strips, sealing rings, etc., and connected to the air pump 16 through wires; It can effectively prevent essential oil from entering the electronic control module 19 and damaging the electronic control module 19.

In this implementation, the equipment is preferably a perfume diffuser, a humidifier, an air purifier, etc.

When it is a fragrance diffuser, the liquid in the bottle body 2 is an essential oil used to produce a fragrance.

The applicant declares that the invention describes the process method of the invention through the above implementation, but the invention is not limited to the above process steps, nor does it mean that the invention must rely on the above process steps to implement. Technical personnel in the technical field of the invention should understand that any improvement of the invention, equivalent replacement of the raw materials selected by the invention and the addition of auxiliary ingredients, the selection of specific methods, etc. fall within the scope of protection and disclosure of the invention.

1. A leak-proof atomizing component, the characteristic is: it comprises an atomizing head for connecting with a peripheral bottle body; the atomizing head is provided with an atomizing core for atomizing the liquid in the bottle body and producing spray; the atomizing head is provided a return liquid hole with a peripheral bottle body;
except the atomizing core, the liquid return hole is the only channel for the peripheral bottle body to connect with the external atmosphere; the liquid return hole is one or more;
when the return liquid hole is only one, the diameter of the return liquid hole is the first preset value, so that the liquid in the peripheral bottle cannot continuously flow out from the return liquid hole when the peripheral bottle is poured;
when the return liquid hole is multiple, the diameter of the return liquid hole is the second preset value, so that when the peripheral bottle body is poured, the liquid in the peripheral bottle body cannot continuously flow out of the return liquid hole due to the existence of liquid tension.

2. The leak-proof atomizing component described in claim 1 is characterized in that the first preset value is less than or equal to 2 mm.

3. The leak-proof atomizing component described in claim 2 is characterized in that the first preset value is less than or equal to 1.5 mm.

4. The leak-proof atomizing component described in claim 3 is characterized in that the first preset value is 0.2˜1.2 mm.

5. The leak-proof atomizing component described in claim 4 is characterized in that the first preset value is 0.5˜1 mm.

6. The leak-proof atomizing component described in claim 1 is characterized in that the second preset value is less than 0.2 mm.

7. The leak-proof atomizing assembly described in claim 1 is characterized in that the atomizing head is provided with an atomizing chamber; the atomizing head is provided with a fog outlet, and the atomizing chamber is conductive to the atmosphere through the fog outlet.

8. The leak-proof atomizing component described in claim 7 is characterized in that the atomizing chamber is provided with an atomizing cover; the atomizing cover divides the atomizing chamber into two connected chambers, and the atomizing mouth and the atomizing core are located on both sides of the atomizing cover.

9. The leak-proof atomizing assembly mentioned in claim 1 is characterized in that the atomizing head is provided with an atomizing seat, and the atomizing core is fixed on the atomizing seat; the atomizing head and the atomizing seat are provided with a gas channel and a liquid channel that leads to the atomizing core; the liquid channel is connected to the peripheral bottle body through a pipeline; the gas channel is connected with a peripheral gas source.

10. The leak-proof atomizing assembly described in claim 1 is characterized in that the liquid return hole is connected with a duct extending to the middle or lower part of the peripheral bottle body.

11. The leak-proof atomizing assembly described in claim 1 is characterized in that the atomizing head is provided with a concave part on one side of the peripheral bottle body; The liquid return hole is arranged in the concave part.

12. The leak-proof atomizing assembly described in claim 11, the leak-proof atomizing component is characterized in that the atomizing core is arranged in the concave part, the liquid return hole is arranged at the lowest place of the concave part, and the top of the concave part is arranged at the lowest place of the corresponding surface of the atomizing head.

13. The leak-proof atomizing assembly described in claim 7 is characterized in that the atomizing head is provided with a baffle ring part; The baffle ring part is arranged on a path where the liquid flows from the return liquid hole to the exit fog gate.

14. The leak-proof atomizing assembly described in claim 13 is characterized in that the baffle ring is arranged on the inside of the atomizing head and near the fog outlet.

15. The leak-proof atomizing component described in claim 13 is characterized in that the baffle ring is raised inward to form a circular platform; The fog outlet is located in the center of the circular platform; The half section of the retaining ring and the half section of the top wall of the atomizing head constitute a “7” font structure.

16. A atomizing device is characterized in that it includes a leak proof atomizing assembly, a bottle body, and an air source as described the items of claim 1; the atomizing head is fixedly connected with the bottle body; the bottle body is used to provide the liquid required for spraying to the atomizing core; the gas source is used to provide compressed gas required for spraying to the atomizing core.

17. The atomizing device in accordance with claim 16 is characterized in that it also includes a shell in which the bottle body is placed.

18. The atomizing device referred to in claim 16 is characterized in that the air pump is located in the housing and below the cylinder.

19. The atomizing equipment described in claim 17 is characterized in that the bottom of the shell is also provided with a base; the base is provided with an electronic control assembly for controlling the air pump.

20. The atomizing device referred to in claim 16 is characterized by the fact that the liquid in the bottle is an essential oil or a disinfectant or mosquito repellent used to produce a fragrance.
